(19) United States
(12) Patent Application Publication
(10) Pub. No.: US 2021/0171464 A1
(43) Pub. Date: Jun. 10, 2021

(54) AMORPHOUS FORM OF PIMAVANSERIN HEMITARTRATE

(71) Applicant: OLON S.P.A., Rodano (MI) (IT)

(72) Inventors: Mattia BERTOLOTTI, Rodano (MI) (IT); Jacopo BONANOMI, Rodano (MI) (IT); Barbara NOVO, Rodano (MI) (IT)

(21) Appl. No.: 16/761,910

(22) PCT Filed: Oct. 29, 2018
(86) PCT No.: PCT/IB2018/058441

§ 371 Date: May 6, 2020

(30) Foreign Application Priority Data
Nov. 6, 2017 (IT) 102017000125959

(51) Int. Cl. C07D 211/58

(52) U.S. Cl. CPC C07D 211/58; C07B 2200/13

(57) ABSTRACT

Disclosed is the amorphous form of pimavanserin* hemitartrate, the process for its preparation, and pharmaceutical formulations containing it.

### FIELD OF INVENTION

The present invention relates to the amorphous form of pimavanserin hemitartrate, the process for its preparation, and pharmaceutical formulations containing it.

Background to the invention N-Azacycloalkyl-N-arylalkyl carbamide compounds have biological activity as serotonin 5-HT receptor inverse agonists. In particular, the compound pimavanserin hemitartrate (N-(4-fluorobenzyl)-N-(1-methylpiperidin-4-yl)-N′ -(4-(2-methyl-propyloxy)phenylmethyl)carbamide hemitartrate) is highly selective for the 5HT_{2A} receptor, with an affinity 40 times greater than for the 5-HT₂B receptor, whereas it has no affinity for the 5-HT_{2C} receptors or dopamine receptors. In view of these characteristics, the action mechanism of pimavanserin hemitartrate differs from those of known antipsychotics. WO 2008144665 discloses that N-(4-fluorobenzyl)-N-(1-methylpiperidin-4-yl)-N′-(4-(2-methylpropyloxyl)phenylmethyl)carbamide hemitartrate can be used for the treatment of psychosis and schizophrenia in patients suffering from Parkinson's disease. Pimavanserin hemitartrate exhibits greater efficacy and fewer side effects than other antipsychotics. Moreover, the administration of pimavanserin in combination with other antipsychotics, such as haloperidol and risperidone, improves the tolerability of the treatment by reducing the incidence of side effects associated with said two medicaments.

Six crystalline forms of pimavanserin hemitartrate, defined as forms A, B, C, D, E and F, are known in the literature, and described in WO2006/037043.

Crystalline form A shows an XRPD diffractogram comprising the following peaks: 18.6 (s), 16.7 (vs), 10.2 (s), 8.2 (m), 7.7 (w), 7.4 (w), 6.5 (w), 6.2 (m), 6.1 (vs), 5.86 (w), 5.14 (m), 5.03 (m), 4,78 (m), 4.69 (m), 4.63 (s), 4.49 (s), 4.44 (vs), 4.35 (m), 4.10 (m), 3.96 (s), 3.66 (m). Crystalline form B shows an XRPD diffractogram comprising the following peaks: 10.2 (s), 8.8 (w), 7.4 (vs), 6.4 (w), 5.91 (vs), 5.46 (w), 4.99 (m), 4.90 (m), 4.62 (m), 4.50 (vs), 4.37 (vs), 4.20 (w), 3.87 (vs), 3.73 (w), 3.58 (m), 3.42 (w), 2.90 (w).

Crystalline form C shows an XRPD diffractogram comprising the following peaks: 12.0 (w), 10.7 (vs), 7.4 (vw), 6.9 (vw), 6.6 (vw), 6.2 (w), 5.86 (m), 5.53 (w), 5.28 (m), 5.16 (m), 4.84 (vs), 4.70 (m), 4.57 (s), 4.38 (m), 4.09 (w), 3.94 (w), 3.77 (s), 3.71 (m), 3.49 (w), 3.46 (w), 3.25 (w), 3.08 (w), 2.93 (w).

Crystalline form D shows an XRPD diffractogram comprising the following peaks: 17.2 (s), 16.0 (m), 10.7 (vw), 9.8 (w), 6.6 (m), 6.1 (s), 6.00 (m), 5.736 (w), 5.33 (w), 5.17 (m), 4.91 (m), 4.64 (s), 4.54 (vs), 4.37 (vs), 4.10 (m), 3.91 (m), 3.84 (m), 367 (w), 3.55 (m), 3.42 (m), 3.32 (w), 3.13 (w), 3.06 (m).

Crystalline form E shows an XRPD diffractogram comprising the following peaks: 17.3 (vs), 16.2 (m), 10.6 (m), 9.8 (m), 8.1 (w), 7.5 (w), 6.6 (m), 6.0 (vs), 5.28 (m), 5.09 (s), 4.90 (m), 4.72 (vs), 4.51 (m), 4.39 (s), 4.26 (s), 4.04 (m), 3.86 (w), 3.70 (w), 3.54 (m), 0.48 (m), 3.02 (w).

Crystalline form F shows an XRPD diffractogram comprising the following peaks: 19.0 (w), 16.0 (m), 13.0 (m), 7.8 (w), 6.4 (m), 6.2 (m), 5.74 (w), 5.29 (w), 5.04 (m), 4.83 (m), 4.62 (m), 4.50 (m), 5.74 (w), 5.39 (w), 5.04 (m), 4.83 (m), 4.62 (m), 4.50 (m), 4.34 (m), 4.24 (vs), 4.05 (m), 3.89 (m), 3.76 (m), 3.58 (w), 3.27 (m).

### SUMMARY OF THE INVENTION

The present invention relates to pimavanserin hemitartrate in amorphous form. The amorphous form of pimavanserin hemitartrate is characterised by an IR spectrum as shown in FIG. 1, a DSC curve as shown in FIG. 2, and an XRPD pattern as shown in FIG. 3.

### DESCRIPTION OF FIGURES

FIG. 1: Infrared spectrum of pimavanserin hemitartrate, amorphous form.

FIG. 2: DSC curve of pimavanserin hemitartrate, amorphous form.

FIG. 3: XRPD pattern of pimavanserin hemitartrate, amorphous form.

FIG. 4: ¹1-1-NMR spectrum of pimavanserin hemitartrate, amorphous form, in d₆-DMSO.

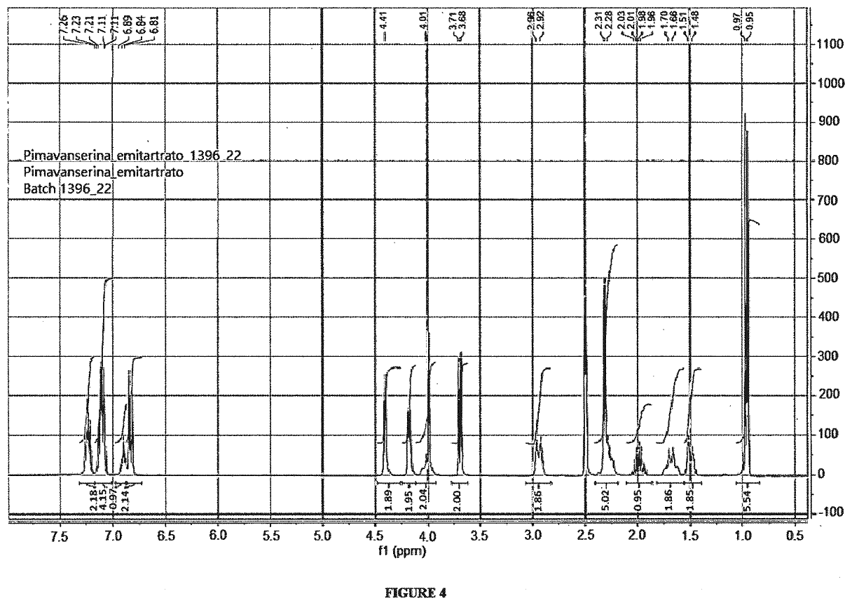

### DETAILED DESCRIPTION OF THE INVENTION

The object of the present invention is pimavanserin hemitartrate (N-(4-fluorobenzyl)-N-(1-methylpiperidin-4-yl)-N′-(4-(2-methylpropyloxy)-phenylmethyl)carbamide hemitartrate) in amorphous form. The amorphous form is characterised by an IR spectrum comprising absorption peaks at 3361.1 cm⁻¹, 2958,9 cm⁻¹, 1610,7 cm⁻¹,1508,3 cm⁻¹, 1470,7 cm⁻¹, 1394,8 cm⁻¹, 1219,5 cm⁻¹, 1172,2 cm⁻¹, 1111,2 cm⁻¹, 1030,3 cm⁻¹, 981,0 cm⁻¹, 817,2 cm⁻¹, 776,5 cm⁻¹.

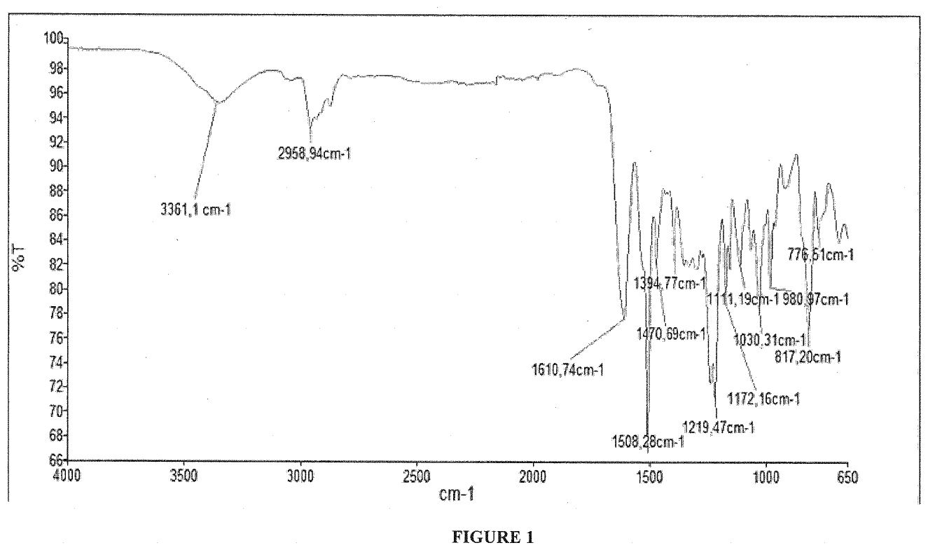

The IR spectrum of the amorphous form is illustrated in FIG. 1. The amorphous form of pimavanserin hemitartrate is also characterised by an endothermic peak at 61.49° C., an exothermic peak at 132.79° C. and an endothermic peak at 167.88° C., detected by DSC (Differential Scanning calorimetry) analysis.

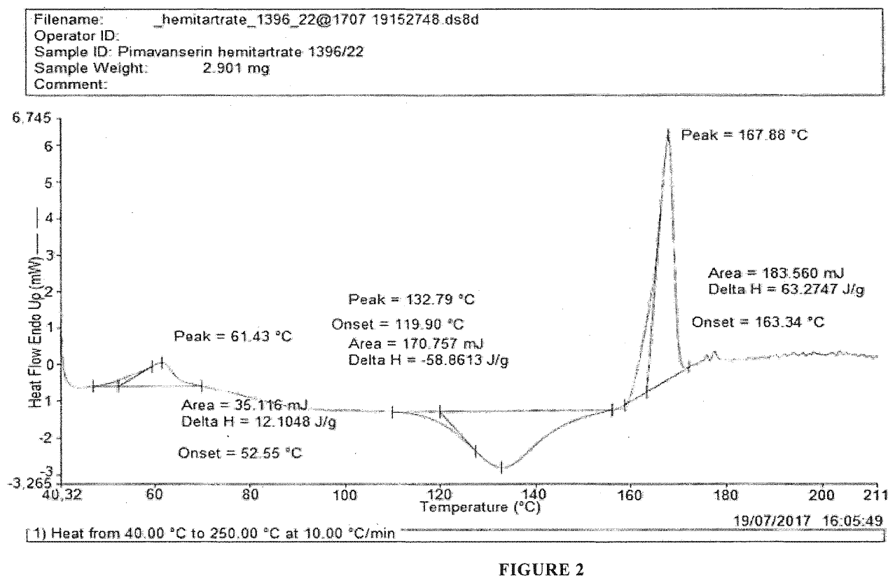

The DSC curve of the amorphous form of pimavanserin hemitartrate is shown in FIG. 2.

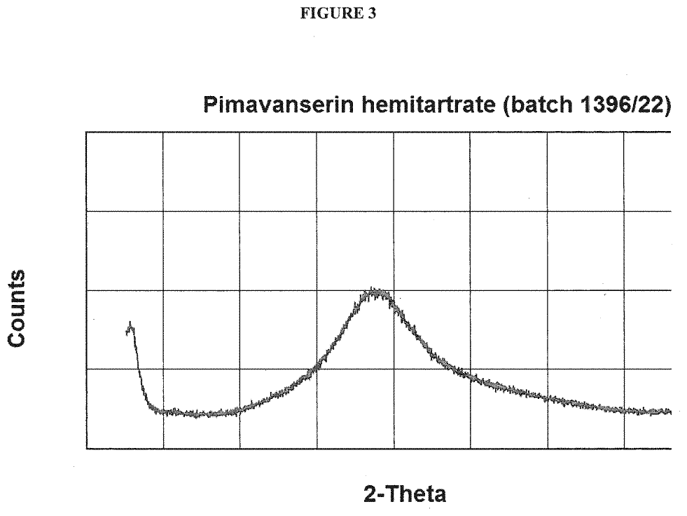

The amorphous form is also substantially characterised by an XRPD pattern as illustrated in FIG. 3.

The amorphous form of pimavanserin hemitartrate exhibits better solubility in various organic solvents and water than the 6 crystalline forms of pimavanserin hemitartrate known from the literature and described in WO2006/037043. The amorphous form of pimavanserin hemitartrate is also stable when exposed to atmospheric humidity, and is not hygroscopic.

A further object of the invention is a process for the preparation of the amorphous form of pimavanserin hemitartrate, comprising the steps of:
a) dissolving pimavanserin hemitartrate in crystalline form in a suitable polar solvent at a temperature ranging between 16° C. and 100° C., preferably between 20° C. and 70° C., and more preferably between 25° C. and 40° C.;
b) rapidly cooling the solution to a temperature ranging between −50° C. and 0° C., preferably between −40° C. and −10° C., and more preferably between −30° C. and −20° C.;
c) maintaining the solution under vacuum for a time ranging between 0 and 72 hours, preferably between 1 hour and 48 hours, and more preferably between 2 hours and 24 hours, at a temperature ranging between −50° C. and 0° C., preferably between −40° C. and −10° C., and more preferably between −30° C. and −20° C.
d) recovering the resulting solid.

The amorphous form of pimavanserin hemitartrate can also be prepared by another procedure which comprises the steps of:
a) dissolving pimavanserin hemitartrate in crystalline form in a suitable polar solvent at a temperature ranging between 16° C. and 100° C., preferably between 20° C. and 70° C., and more preferably between 25° C. and 40° C.;
b) evaporating the solvent on a thin layer at a temperature ranging between −20° C. and 120° C., preferably between −10° C. and 100° C., and more preferably between 5° C. and 60° C., at atmospheric pressure, until complete evaporation of the solvent;
c) recovering the resulting solid.

The pimavanserin hemitartrate in crystalline form used as starting product in the processes reported above is preferably one of the crystalline forms described in WO2006/037043.

The polar solvent used for the dissolution (step a) in the processes described above is preferably selected from the group consisting of water, methanol, ethanol, 1-butanol, 1-propanol, isopropanol, methyl ethyl ketone, acetone, ethyl acetate, tetrahydrofuran, dioxane, methyl tert-butyl ether, acetonitrile, isopropyl acetate, isobutyl acetate, dimethylsulphoxide, dimethylformamide and dichloromethane, or a mixture thereof More preferably the solvent is water, methanol, dichloromethane, dimethylformamide or dimethylsulphoxide or a mixture thereof. The most preferred solvent is water.

The solid recovered at the end of the evaporation process is the amorphous form of pimavanserin hemitartrate, which shows the IR spectrum, DSC curve and XRPD diffractogram shown in FIGS. 1, 2 and 3 respectively.

A further object of the invention is a pharmaceutical formulation comprising the amorphous form of pimavanserin hemitartrate and at least one pharmaceutically acceptable carrier and/or excipient.

The formulations according to the invention can be used in the treatment of psychosis and schizophrenia, in particular in patients suffering from Parkinson's disease.

### EXAMPLES

IR spectra were recorded with a Perkin Elmer Spectrum 1000 IR instrument. The sample was prepared as a KBr pellet. The spectrum was recorded by performing 16 scans at a resolution of 4 cm⁻¹.

DSC curves were recorded with a Perkin Elmer Pyris 1 instrument, and 3-5 mg of material were used to prepare the samples. The scans were conducted at the rate of 10° C. a minute.

NMR spectra were recorded with a Varian Mercury 300 instrument in DMSO at 25° C., 16 scans being performed.

XRPD spectra were recorded with a Bruker D2 instrument which uses the following parameters: Wavelength CuKα (λ=15419 A)—Energy 30 KV—Stepsize: 0.02°-2θRange: 2.6°-40°.

### Example 1

Pimavanserin hemitartrate (25.0 g) was suspended in 125 ml of water, and the suspension was heated at T=40° C. until completely dissolved. The solution was cooled at −30° C. for 24 hours.

The solution was placed in a freeze dryer at −30° C. under vacuum, and a white solid was obtained after 24 hours. The (amorphous) product shows an IR spectrum, DSC curve and XRPD pattern as shown in FIGS. 1-3 respectively.

### Example 2

Pimavanserin hemitartrate (25.0 g) was suspended in 100 ml of water, and the suspension was heated at T=40° C. until completely dissolved. The solution was stored in a crystalliser at 40° C. at atmospheric pressure until the solvent had completely evaporated.

The resulting (amorphous) white solid shows an IR spectrum, DSC curve and XRPD pattern as shown in FIGS. 1-3 respectively.

1. Amorphous solid form of pimavanserin hemitartrate.

2. The amorphous solid form according to claim 1, characterised by an IR spectrum comprising absorption peaks at 3361.1 cm⁻¹, 2958.9 cm⁻¹, 1610.7 cm⁻¹, 1508.3 cm^{″1}, 1470.7 cm⁻¹, 1394.8 cm⁻¹, 1219.5 cm⁻¹, 1172.2 cm⁻¹, 1111.2 cm⁻¹, 1030.3 cm ⁻¹, 981.0 cm⁻¹, 817.2 cm⁻¹ and 776.5 cm⁻¹.

3. The amorphous solid form according to claim 1, characterised by the following IR spectrum:

[symbol]

4. The amorphous solid form according to claim 1, characterised by an endothermic peak at 61.49° C., an exothermic peak at 132.79° C. and an endothermic peak at 167.88° C., detected by DSC analysis.

5. The amorphous solid form according to claim 1, characterised by the following DSC curve:

[symbol]

6. The amorphous solid form according to claim 1, characterised by the following XRPD diffractogram:

[symbol]

7. A process for preparing the amorphous solid form of pimavanserin hemitartrate according to claim 1, comprising the steps of:
a) dissolving pimavanserin hemitartrate in crystalline form in a suitable polar solvent at a temperature ranging between 16° C. and 100° C.;
b) rapidly cooling the solution to a temperature ranging between −50° C. and 0° C.;
c) keeping the solution under vacuum at a temperature ranging between −50° C. and 0° C. for a time of between 0 and 72 hours;
d) recovering the resulting solid.

8. A process for preparing the amorphous solid form of pimavanserin hemitartrate according to claim 1, comprising the steps of:
a) dissolving pimavanserin hemitartrate in crystalline form in a suitable polar solvent at a temperature ranging between 16° C. and 100° C.;
b) evaporating the solvent on a thin layer at a temperature ranging between −20° C. and 120° C. at atmospheric pressure until the solvent has completely evaporated;
c) recovering the resulting solid.

9. The process according to claim 7, wherein the polar solvent used for the dissolution (step a) is selected from the group consisting of water, methanol, ethanol, 1-butanol, 1-propanol, isopropanol, methyl ethyl ketone, acetone, ethyl acetate, tetrahydrofuran, dioxane, tert-butyl methyl ether, acetonitrile, isopropyl acetate, isobutyl acetate, dimethylsulphoxide, dimethylformamide and dichloromethane, or a mixture thereof.

10. The process according to claim 9, wherein the polar solvent used for the dissolution (step a) is selected from the group consisting of water, methanol, dichloromethane, dimethylformamide and dimethylsulphoxide.

11. The process according to claim 10, wherein the polar solvent used for the dissolution (step a) is water.

12. Pharmaceutical formulation comprising the amorphous form of pimavanserin hemitartrate according to claim 1 and at least one pharmaceutically acceptable carrier and/or excipient.

13. The formulation according to claim 12, for use in the treatment of psychosis and schizophrenia.

14. The amorphous solid form according to claim 2, characterised by the following IR spectrum:

[symbol]

15. The amorphous solid form according to claim 2, characterised by an endothermic peak at 61.49° C., an exothermic peak at 132.79° C. and an endothermic peak at 167.88° C., detected by DSC analysis.

16. The amorphous solid form according to claim 3, characterised by an endothermic peak at 61.49° C., an exothermic peak at 132.79° C. and an endothermic peak at 167.88° C., detected by DSC analysis.

17. The amorphous solid form according to claim 2, characterised by the following DSC curve:

[symbol]

18. The amorphous solid form according to claim 3, characterised by the following DSC curve:

[symbol]

19. The amorphous solid form according to claim 4, characterised by the following DSC curve:

[symbol]

20. The amorphous solid form according to claim 2, characterised by the following XRPD diffractogram: [symbol]
